(19) United States
(12) Patent Application Publication
(10) Pub. No.: US 2011/0028412 A1
(43) Pub. Date: Feb. 3, 2011

(54) Herbal enhanced analgesic formulations

(75) Inventors: John V. Cappello, King of Prussia, PA (US); Lawrence Durst, Philadelphia, PA (US)

Correspondence Address: Lawrence J. Shurupoff, 1665 Topanga Lane, Delray Beach, FL, 33484, US

(73) Assignee: Cappellos, Inc.

(21) Appl. No.: 12/804,972

(22) Filed: Aug. 3, 2010

Related U.S. Application Data
(60) Provisional application No. 61/273,254, filed on Aug. 3, 2009.

(51) Int. Cl. A61K 31/7034; A61P 25/04

(52) U.S. Cl. 514/25

(57) ABSTRACT

The analgesic properties of L-tryptophan and 5-HTP can be safely enhanced with the coadministration of salacin. Salacin can be effectively provided in the form of white willow bark along with other ingredients to further enhance the formulation's analgesic effect. As salacin can cause the loss of vitamin C in humans, the formulation advantageously includes a supplemental amount of vitamin C.

### CROSS-REFERENCE TO RELATED APPLICATIONS

This application claims the benefit and priority of provisional patent application No. 61/273,254 filed Aug. 3, 2009, entitled Herbal Enhanced Analgesic Formulations, and which is incorporated herein in its entirety by reference.

### BACKGROUND AND SUMMARY

Intractable chronic pain can be eliminated or relieved through administration of a formulation including L-tryptophan in combination with the additional ingredients fructose, pyridoxine and niacinamide. Each additional ingredient either promotes the transport of L-tryptophan from the blood plasma across the blood-brain barrier into the brain or promotes the conversion of L-tryptophan within the brain to the neurotransmitter serotonin.

The production of the neurotransmitter serotonin can also be increased through administration of a therapeutic composition which includes L-tryptophan in combination with a salicylate, pyridoxine, niacin and a carbohydrate such as fructose. Both the absolute free fraction and the relative amount of the albumin-bound fraction of serum L-tryptophan are increased so that transport of L-tryptophan from the blood plasma across the blood-brain barrier into the brain is increased. Once within the brain, L-tryptophan is converted to serotonin which promotes pain relief and provides other beneficial effects.

The ability to proceed to market with a product incorporating a salicylate like aspirin has been impeded due to the need and cost for what is known as a New Drug Application or NDA. Previously, work to proceed with a product based upon L-tryptophan was impeded by the Food and Drug Administration (FDA) findings of a contaminant in L-tryptophan that caused myalgias and death. It therefore was once desirable to avoid the use of aspirin and L-tryptophan type compounds in order to expedite market entry of a safe and effective analgesic.

L-tryptophan has now become once again commercially and economically available and may now be used in dietary formulations including pain relief formulations.

Alternatives to analgesics which require an NDA include herbal formulations which generally do not require an NDA. One example of an herbal analgesic is found in U.S. Pat. No. 6,312,736 which discloses an herbal composition used to relieve pain and other symptoms associated with migraines and other types of headaches. This herbal composition can include white willow bark extract, kava kava root extract, feverfew extract, ginger root extract, guarana extract, and vitamin B6. The herbal composition may be combined with liposomes to carry the composition. The result is an herbal composition that can be applied sublingually for pain relief. This formulation is significantly different from the formulations disclosed below in accordance with the subject disclosure.

### SUMMARY

The present disclosure is directed to the use of willow bark to increase the analgesic effect of L-tryptophan and 5-HTP. Although willow bark has been used for pain relief, there are no known formulations for increasing the production of serotonin by replacing a salicylate such as aspirin with willow bark. It is believed that such replacement causes several major beneficial factors to occur that have previously gone unrecognized. The first is that willow bark in combination with other pain relievers can enhance an analgesic effect in humans. Second, the use of willow bark enables the introduction to the market of a safer alternative than aspirin without negative gastro-intestinal effects. Third, no advance in the formulation of pain relievers as proposed herein has been located in the market or in the literature wherein willow bark or its pain relieving component salacin have been combined with other ingredients to promote the production of serotonin and the enhanced relief of pain.

Ingestion of salacin causes a release of tryptophan from its binding site on serum albumin and results in the presence of a free, unbound fraction of L-tryptophan within the blood. This free fraction of L-tryptophan is believed to control the concentration of L-tryptophan in the brain, thereby increasing the brain's production of serotonin.

Instead of L-tryptophan, 5-hydroxytryptophan or “5-HTP” can also be used as a safe alternative to L-tryptophan or in comination with L-tryptophan. That is, 5-hydroxytryptophan or 5-HTP is a naturally-occurring amino acid, a precursor to the neurotransmitter serotin and an intermediate in tryptophan metabolism. It is marketed in the United States and other countries as a dietary supplement for use as an antidepressant, appetite suppressant, and sleep aid. As described below, white willow bark, its pain relieving component salacin, as well as other herbs, can be used to enhance the pain-relieving effects of 5-HTP. L-tryptophan, however, is acceptable as an alternative to 5-HTP or in combination with 5-HTP for use as a pain reliever when combined with any of the ingredients and formulations described below.

### DESCRIPTION OF REPRESENTATIVE EMBODIMENTS

White willow bark, also known as salix alba, white willow, and willow bark is a tree native to Europe and Asia. The name “white willow” comes from the color of the leaves, which are covered with fine white hairs. The use of white willow bark medicinally goes back to the ancient Egyptians who used white willow to treat inflammation. The Greek physician Hippocrates wrote about white willow's medicinal uses in the 5th century B.C.

In 1829, scientists in Europe identified what was believed to be the active ingredient in white willow bark—a compound called salicin, although other anti-inflammatory constituents also are present in white willow bark. Public demand grew rapidly for salacin.

Extracting salicin from herbs was considered to be expensive and time-consuming, so a synthetic salicylic acid version was developed in Germany in 1852 and quickly became the treatment of choice. Salicin is converted in the body to salicylic acid.

The problem was that salicylic acid is harder on the stomach than salicin. At therapeutic doses, people using the synthetic salicyclic acid developed stomach ulcers and bleeding.

The German company Bayer eventually created a synthetic, less harsh, derivative of salicylic acid called acetylsalicylic acid (ASA), and mass-produced it under the name aspirin. Despite this, aspirin is still known for irritating the stomach lining. Replacing aspirin with salacin can alleviate this problem.

Additional herbs, as identified below, can be combined with a formulation of L-tryptophan, and/or 5-HTP and one or more of niacin or nicotinamide, pyridoxine, fructose, vitamin C and white willow bark (salacin) to achieve enhanced pain relief in a safe, “easy on the stomach”, oral capsule or other formulation. While these additional herbs are associated with relief or treatment of certain maladies, any of the following combinations of herbs can be used in combination with a base formulation of L-tryptophan and/or 5-HTP, niacin or nicotinamide, pyridoxine, fructose, vitamin C and white willow bark or any extract of white willow bark.

For example, cramps and spasms can be treated with angelica, cramp bar, kava, rosemary, and/or valerian root. Nerve pain can be treated with capsaicin, chamomile, gotu kola, licorice, and white willow. Back pain can be treated with hops, wood betony, and/or passionflower. Migraines can be treated with feverfew, linden, and/or skullcap and headaches can be treated with peppermint and/or spearmint. Any of these herbs can be used with the base formulation described below.

Other herbs that are also useful in pain relief and which can be combined with one or more of L-tryptophan and/or 5-HTP, niacin or nicotinamide, pyridoxine, fructose, vitamin C and white willow bark (the “base formulation”) are set forth below.

Hot peppers such as cayenne pepper (capsilum) triggers the release of the body's own pain-relieving endorphins and also includes salicylates.

Cramp bark and black haw can be used for the treatment of spasmodic pain. Both cramp bark (Viburnum opulus) and black haw (Viburnum prunifolium) can be used to treat both menstrual pain, muscle spasm and arthritic pain. These plants contain the antispasmodic and muscle-relaxing compounds esouletin and scopoletin. These antispasmodic constituents are best extracted with alcohol, so tinctures rather than teas should be used. Black haw also contains aspirin-like compounds. Equal parts of cramp bark and black haw tinctures in amounts between 1 and 4 droppers every two or three hours can be taken for up to three days in combination with the subject L-tryptophan and/or 5-HTP base formulation.

Menthol and camphor can be added to the base formulation to help ease the muscle tightness that contributes to many back pains. Menthol is a natural constituent of plants in the mint family, particularly peppermint (menthe piperita) and spearmint, although the aromatic oils of other mints contain menthol as well. Camphor occurs in spike lavender, hyssop and coriander.

Ginger can be added to the base formulation to treat various sorts of pain. Ginger contains 12 different aromatic anti-inflammatory compounds, including some with mild aspirin-like effects. A fresh ginger root (about the size of a thumb) can be cut into thin slices and placed in a quart of water. The water is then brought to a boil, and then simmered on the lowest possible heat for thirty minutes in a covered pot, then cooled for thirty more minutes and then strained. About ½ to 1 cup of the strained liquid is then taken orally along with the base formulation.

Rosemary can also be added to the subject base formulation. Drinking rosemary tea for pain is a known remedy. Rosemary leaf contains anti-inflammatory substances including camosol, oleanolic acid, rosmarinic acid, and ursolic acid. Carnosol acts on the same anti-inflammatory pathways as both steroids and aspirin. Rosmarinic acid acts through at least two separate anti-inflammatory biochemical pathways and ursolic acid, which makes up about 4 percent of the plant by weight, has been shown in animal trials to have anti-arthritic effects.

Epsom salt is reputed to have healing properties. Epsom salt is primarily magnesium sulfate. The heat of an Epsom salt bath can increase circulation and reduce the swelling of arthritis, and the magnesium can be absorbed through the skin. Magnesium is one of the most important minerals in the body, participating in at least 300 enzyme systems. Magnesium has both anti-inflammatory and anti-arthritic properties. Taking an external Epsom salt bath while also ingesting the base formulation can provide an enhanced analgesic effect.

Angelica formulations (a) comprising tinctures and/or diluted extracts of one, several or all of the following herbs selected from Bellis Perennis, Calendula Officinalis, Hamamelis Virginiana, Arnica Montana, Hypericum Perforatum, Aconitum Napellus, Ledum Palustre, Bryonia Alba and Ruta Graveolens; or (b) including as active ingredients, tinctures and/or diluted extracts from herbs selected from Bellis Perennis, Calendula Officinalis, Hamamelis Virginiana, Arnica Montana, Hypericum Perforatum, Aconitum Napellus, Ledum Palustre, Bryonia Alba and Ruta Graveolens can be included with the subject base formulation. These potentized homeopathics can be provided in a penetrating base, preferably a clear gel base in combination with oral administration of the base formulation.

A therapeutic composition comprising oregano oil, laurel oil, and myrtle oil useful in alleviating pain and discomfort associated with arthritis, migraines, bronchitis, soft tissue injuries, muscle aches and pains and neck and back pains and strains in humans, as well as upper respiratory, joint and shin ailments in animals can also be added to the subject base formulation.

An externally applicable analgesic composition including an extract derived from sumac leaves, sassafrass root, oak tree bark and an alcohol component, combined with benzocaine, procaine and menthol components can supplement the oral administration (ingestion) of subject base formulation. The topical application of this composition has proven effective for the temporary relief of pain and stiffness associated with arthritis, bursitis, muscle cramp and other aches and pains.

Also disclosed for pain are compositions containing either aspartame or monosodium glutanate combined with an amino acid and added to the base formulation.

Those familiar with the art may find ways to incorporate any one or more of the herbs mentioned above into the subject base formulation including 5-HTP or L-tryptophan and/or any one or more of niacinamide, pyridoxine, fructose, vitamin C and willow bark or salicin. An oral capsule of such a formulation is one desired form of administration.

Fructose is included in each capsule as a preferred source of carbohydrate to achieve insulin/LNAA/tryptophan effect. That is, not only is tryptophan and 5-HTP carried by this transport mechanism, but other selected amino acids, called large electrically neutral amino acids (LNAA's) are carried across the blood-brain barrier as well.

A lowered pain threshold is enabled with L-tryptophan or 5-HTP being able to pass the blood brain barrier in combination with fructose, niacin and pyridoxine (vitamin B-6) thereby effectively increasing serotonin and enhancing the analgesic effectiveness of the salicin derived from willow bark.

Since willow bark and salicylates such as aspirin can also cause the loss of vitamin C, another important focus of the formulation is to include vitamin C or ascorbic acid as a component of the base formulation in an amount from 50 mg to 500 mg. to replace any vitamin C or ascorbic acid loss caused by ingestion of salacin and/or willow bark.

Since analgesics are commonly used to treat cold and flu symptoms, and it is known that 9 to 24 mg. of elemental zinc may add certain benefits, the use of zinc such as in the form of a zinc salt such as zinc gluconate may also be added to the base formulation for effectively treating cold and flu symptoms.

### EXAMPLE

While the weight of each ingredient listed below could vary up to approximately 50% more, the base formulation for an effective analgesic single dosage for a typical human patient is as follows:

(1) L-tryptophan or 5-HTP or a combination thereof, up to about 250 mg; (2) niacinamide (niacin or nicotinamide), a natural vitamin, up to about 25 mg; (3) pyridoxine (vitamin B6), a natural vitamin, up to about 25 mg; (4) fructose, a natural sugar, up to about 25 mg, (5) salicin, such as provided in willow bark (white willow bark) in an amount sufficient to provide up to about 500 mg of salicin and vitamin C in an amount up to about 500 mg.

Another example of an effective analgesic formulation is set forth below, with the weight of each ingredient variable within a range of plus or minus 50%, as broadened and defined by the term “about”. Zinc gluconate is optional.

salicin about 250 mg.
L-tryptophan about 100 mg.
niacin about 10 mg.
vitamin B-6 about 10 mg.
fructose about 25 mg.
vitamin C about 100 mg.
zinc gluconate about 100 mg. (optional)

In the examples, salacin can be provided in the form of (white) willow bark extract. Such an extract is commercially available with the concentration of salacin ranging from 15% to 95% by weight. The concentration of salacin in extracts is not particularly critical as long as the prescribed weight of salacin is administered to one in need of pain relief.

While the base formulation is susceptible to alternative constructions, certain embodiments thereof have been described above in detail. It should be understood, however, that there is no intention to limit the disclosure to the specific form or embodiments disclosed, but on the contrary, the intention is to cover all modifications, alternative constructions, and equivalents falling within the spirit and scope of the disclosure.

For example, the claimed formulation may be in the form of powders, tablets, capsules, lozenges, and bicarbonated versions to produce a seltzer drink, a ready to use liquid form in water or other palatable medium or topical gel or cream. As used in the claims, the term “about” means a variance of fifty percent (50%).

What is claimed is:

1. A analgesic formulation comprising:
salacin;
at least one of L-tryptophan and 5-HTP;
niacinamide;
pyridoxine;
fructose; and
vitamin C.

2. The formulation of claim 1, wherein said salicin is provided in the form of at least one of white willow bark and extracts of white willow bark.

3. The formulation of claim 1, wherein said salicin comprises up to about 500 mg of salacin.

4. The formulation of claim 1, wherein said at least one of said L-tryptophan and 5-HTP comprises up to about 250 mg of said at least one of said L-tryptophan and 5-HTP.

5. The formulation of claim 1, wherein said niacinamide comprises up to about 25 mg of at least one of niacin and nicotinamide.

6. The formulation of claim 1, wherein said pyridoxine comprises up to about 25 mg of pyridoxine.

7. The formulation of claim 1, wherein said fructore comprises up to about 25 mg of fructose.

8. The formulation of claim 1, wherein said vitamin C comprises up to about 100 mg. of vitamin C.

9. The formulation of claim 1, further comprising zinc.

10. An analgesic formulation, comprising:
up to about 500 mg salacin;
up to about 250 mg L-tryptophan;
up to about 25 mg niacinamide;
up to about 25 mg pyridoxine;
up to about 25 mg fructose; and
up to about 500 mg vitamin C.

11. The formulation of claim 10, further comprising up to 24 mg of zinc.

12. The formulation of claim 10, wherein said salacin comprises about 250 mg of salacin.

13. The formulation of claim 10, wherein said salacin is provided in the form of white willow bark.

14. The formulation of claim 10, wherein said L-tryptophan comprises about 100 mg of L-tryptophan.

15. The formulation of claim 10, wherein said niacinamide comprises about 10 mg of niacin.

16. The formulation of claim 10, wherein said fructose comprises about 25 mg of fructose.

17. The formulation of claim 10, wherein said vitamin C comprises about 100 mg of vitamin C.

18. The formulation of claim 11, wherein said zinc is provided in the form of about 100 mg of zinc gluconate.

19. A method of relieving pain in a human, comprising:
orally administering to the human a formulation comprising salacin, at least one of L-tryptophan and 5-HTP, niacinamide, pyridoxine, fructose and vitamin C.

20. The method of claim 19, wherein said salacin is administered in the form of at least one of white willow bark and extracts of white willow bark.
